(19) United States
(12) Patent Application Publication
(10) Pub. No.: US 2006/0194984 A1
(43) Pub. Date: Aug. 31, 2006

(54) Methods of making pravastatin sodium

(76) Inventors: Vilmos Keri, Debrecen (HU); Edit Nagyne Arvai, Debrecen (HU); Zoltan Czovek, Debrecen (HU); Adrienne Kovacsne-Mezei, Debrecen (HU); Istvan Viktor Katai, Debrecen (HU); Csilla Nemethne Racz, Tiszavasvari (HU)

Correspondence Address: KENYON & KENYON LLP, ONE BROADWAY, NEW YORK, NY, 10004, US

(21) Appl. No.: 11/351,468

(22) Filed: Feb. 9, 2006

Related U.S. Application Data
(60) Provisional application No. 60/651,738, filed on Feb. 9, 2005.

(51) Int. Cl. C07C 69/66

(52) U.S. Cl. 560/185

(57) ABSTRACT

The invention encompasses a new crystalline form of pravastatin sodium characterized by X-ray powder diffraction peaks at 3.3, 3.9, 5.4, 6.4, 16.8, and 17.5 degrees two-theta, ±0.1 degrees two-theta and to methods of forming the crystalline form of the present invention and methods of making pravastatin Form B and Form D.

### RELATED APPLICATIONS

This application claims the benefit of U.S. Provisional Application No. 60/651,738, filed Feb. 9, 2005, hereby incorporated by reference.

### FIELD OF THE INVENTION

The invention is directed to methods of making pravastatin sodium by a production scale drying process and a novel form of pravastatin sodium made by the method.

### BACKGROUND OF THE INVENTION

Pravastatin is the common medicinal name of the chemical compound [1S-[1α(β*, δ*)2α,6α,8β(R*),8aα]]-1,2,6,7,8,8a-hexahydro-β, δ,6-trihydroxy-2-methyl-8-(2-methyl-1-oxobutoxy)-1-naphthalene-heptanoic acid. (CAS Registry No. 81093-370.) The molecular structure of pravastatin in free acid form is represented by Formula (I):

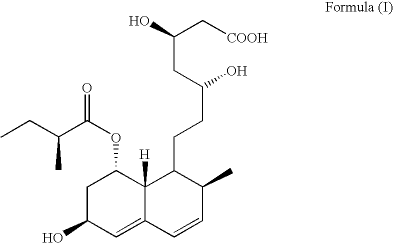

Pravastatin exhibits an important therapeutic advantage over other statins. Pravastatin selectively inhibits cholesterol synthesis in the liver and small intestine but leaves cholesterol synthesis in the peripheral cells substantially unaffected. Koga, T. et al., Biochim. Biophys. Acta, 1045, 115-120 (1990). The selectivity appears to be due, in part, to the presence of a hydroxyl group at the C-6 position of the hexahydronaphthalene nucleus. The C-6 position is occupied by a hydrogen atom in compactin and a methyl group in lovastatin. Pravastatin is less able to permeate the lipophilic membranes of peripheral cells than the other more lipophilic congeners. Serajuddin et al., J. Pharm. Sci., 80, 830-34 (1991). Also, the limited mobility of pravastatin is thought to account for its more localized action in the liver and intestine.

International application No. WO 01/43723 discloses polymorphs of pravastatin sodium and preparation thereof.

Because polymorphs of the same compound may affect stability (i.e., shelf-life) solubility, dissolution rate (determining bioavailability), compressibility, or density, alternative polymorphs are necessary to provide alternatives to the medicinal chemist when preparing formulations.

The solid state physical properties can be influenced by controlling the conditions under which pravastatin sodium is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in an aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is also a need in the art for new processes for obtaining the different polymorphic forms.

The present invention relates to the solid state physical properties of pravastatin sodium.

### SUMMARY OF THE INVENTION

The invention is directed to a new crystalline form of pravastatin sodium, and methods of making this crystalline form, and processes for making crystalline forms B, and D.

In one embodiment, the present invention provides a crystalline form of pravastatin sodium characterized by data selected from a group consisting of: an X-ray powder diffraction with peaks at 3.3, 3.9, 5.4, 6.4, 16.8, and 17.5±0.1 degrees two-theta, an FT-IR spectrum with peaks at 1157, 1181, 1570, and 1731±2 cm⁻¹ and DSC thermogram having a broad endotherm at about 108° C., and an endotherm at about 176° C. The crystalline form of the present invention may be a hydrate. Preferably, the crystalline form of the present invention is a monohydrate.

In another embodiment, the present invention provides a process for preparing the crystalline form of the present invention comprising suspending wet crystals of pravastatin sodium Form L in acetone, reducing the water content to about 3% to about 7% by weight, and drying the crystals at a temperature of about 35° C. to about 45° C.

In one embodiment, the present invention provides a process for preparing pravastatin sodium Form B comprising suspending wet crystals of pravastatin sodium Form L in acetone, reducing the water content to about 3% to about 7% by weight and drying the crystals at a temperature of about 60° C.

In another embodiment, the present invention provides a process for preparing pravastatin sodium Form B comprising providing dried crystals of pravastatin sodium Form D, suspending the dried crystals of Form D in a solvent mixture of water and acetone, reducing the water content to about 3% to about 7% by weight and drying the crystals.

In another embodiment, the present invention provides a process for preparing pravastatin sodium Form D comprising providing wet crystals of pravastatin sodium Form L, drying the wet crystals at a temperature of about 50° C. to about 70° C. under a pressure of between about reduced pressure to about atmospheric pressure.

In another embodiment, the present invention provides pharmaceutical compositions comprising the crystalline form of the present invention.

In another embodiment, the present invention provides a process for preparing a pharmaceutical formulation comprising combining the crystalline form of the present invention with at least one pharmaceutically acceptable excipient.

In another embodiment, the present invention provides the use of the crystalline form of the present invention for the manufacture of a pharmaceutical composition.

### BRIEF DESCRIPTION OF THE DRAWINGS

FIG. 1 illustrates the powder X-ray diffraction pattern of the crystalline form of the present invention.

FIG. 2 illustrates the infrared absorption spectrum of the crystalline form of the present invention.

FIG. 3 illustrates the DSC curve of the crystalline form of the present invention.

FIG. 4 illustrates the TGA thermogram spectrum of the crystalline form of the present invention.

FIG. 5 is a polarized light microscopical picture of the crystalline form of the present invention.

### DETAILED DESCRIPTION OF THE INVENTION

As used herein the term “substantially pure” means having less than 10% by weight of other crystal forms.

As used herein the term “wet crystals” refers to crystals having a water content of at least about 7% by weight, and preferably having a water content of about 11% to about 15% by weight.

As used herein the term “dried crystals” refers to crystals having less than about 2% of water by weight.

As used herein the term “reduced pressure” refers to a pressure of about of about 5 mm Hg to about 600 mm Hg.

Crystalline pravastatin sodium Form B is characterized by X-ray powder diffraction peaks at 3.6, 6.1, 6.6, 9.0, 9.6 and 10.1±0.2 degrees two-theta and by additional peaks at about 16.4, 16.8 and 18.6±0.2 degrees two-theta. Pravastatin sodium Form B may be further characterized by an FT-IR spectrum with peaks at 614, 692, 739, 824, 842, 854, 868, 901, 914, 936, 965, 1011, 1028, 1039, 1072, 1091, 1111, 1129, 1149, 1161, 1185, 1232, 1245, 1318, 1563, 1606, 1711 and 1730±2 cm⁻¹.

Crystalline pravastatin sodium Form D is characterized by X-ray powder diffraction peaks at 3.6, 6.3, 9.8 and 17.1±0.2 degrees two-theta. Pravastatin sodium Form D may be further characterized by an FT-IR spectrum with peaks at 824, 843, 854, 914, 939, 965, 1013, 1041, 1079, 1091, 1157, 1186, 1266, 1566, 1606 and 1728±2 cm^{-1.}

Crystalline pravastatin sodium Form L is characterized by X-ray powder diffraction peaks at 16.6, 17.6 and 18.5±0.2 degrees two-theta and by additional peaks at about 4.5, 5.0, 9.0, 10.1, 12.3, 13.4, 15.0, 19.5, 20.2, 21.2 and 22.7±0.2 degrees two-theta. The preparation of crystalline pravastatin sodium Form L is disclosed in the International publication WO 01/43723.

The invention is directed to a new crystalline form of pravastatin sodium, and methods of making this crystalline form, and methods of making pravastatin sodium crystals forms B and D.

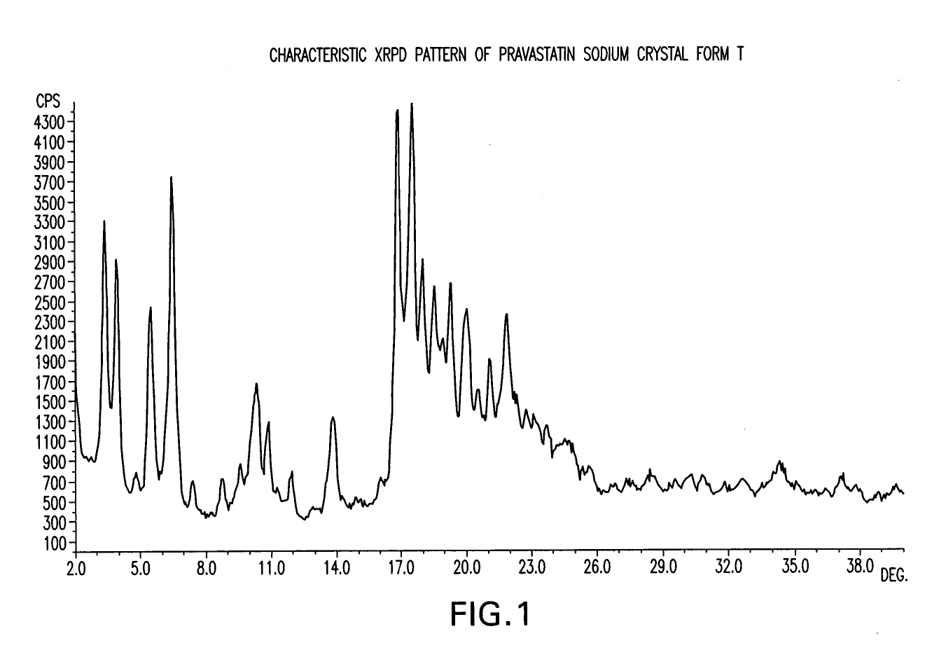

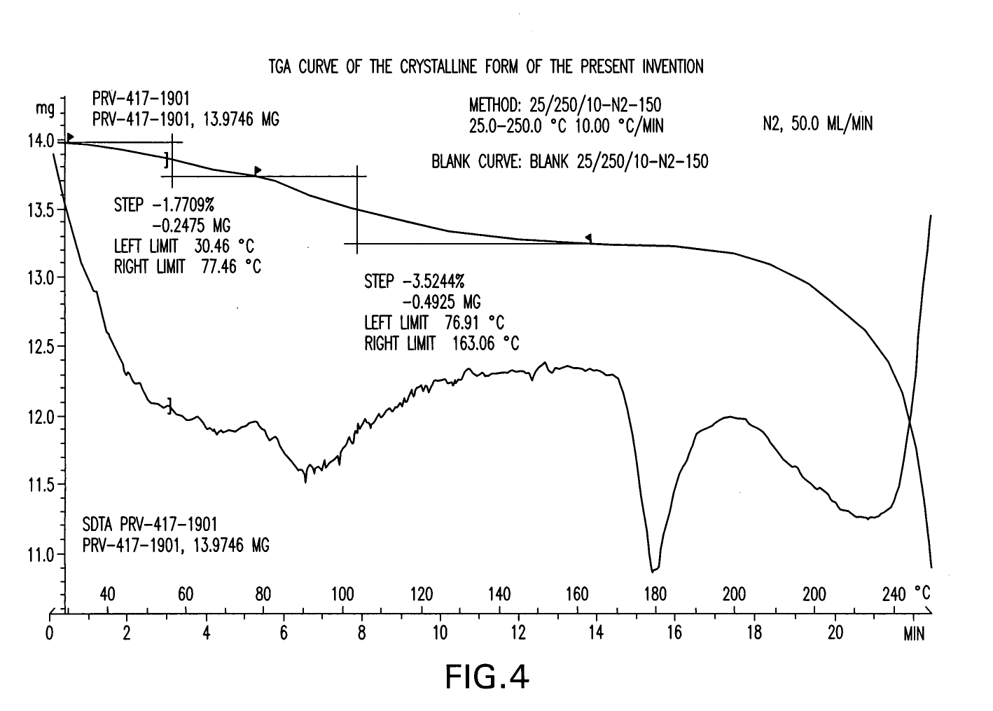

In one embodiment, the present invention provides crystalline form of pravastatin sodium characterized by data selected from a group consisting of: an X-ray powder diffraction with peaks at 3.3, 3.9, 5.4, 6.4, 16.8, and 17.5±0.1 degrees two-theta, an FT-IR spectrum with peaks at 1157, 1181, 1570, and 1731±2 cm⁻¹ and a DSC thermogram having a broad endotherm at about 108° C., and an endotherm at about 176° C. The crystalline form may be a hydrate. Preferably, the crystalline form is a monohydrate. The crystalline form may contain about 2.8% to about 4.2% of water by weight as measured by Karl Fisher or TGA. Preferably, the crystalline form contains about 3.8% by weight. The crystalline form may be further characterized by X-ray powder diffraction peaks at 10.2, 13.8, 18.0, 19.3, 19.5, and 21.8 degrees two-theta, ±0.1 degrees two-theta. The crystalline form may be further characterized by an FT-IR spectrum with peaks at 722, 823, 843, 964, 1014, 1037, 1080, 1109 and 1263±2 cm⁻¹±2 cm⁻¹. Appropriate PXRD, FTIR, DSC and TGA figures correspond to FIGS. 1, 2, 3 and 4.

Preferably, the crystalline form of the present invention is substantially pure.

Preferably, the crystalline form of the present invention has a particle size of less than about 250 μm. Preferably, the crystals of the crystalline form of the present invention have irregularly shaped stacked plates, and fractured edges and a rough surface. A polarized light microscopical picture of the crystalline form of the present invention is provided in FIG. 5.

In another embodiment, the present invention provides a process for preparing the crystalline form of the present invention comprising suspending wet crystals of pravastatin sodium Form L in acetone, reducing the water content to about 3% to about 7% by weight, and drying the crystals at a temperature of about 35° C. to about 45° C. Preferably, the volume of acetone used is of about 15 times the mass of crystals to be suspended. Preferably, the reduction of the water content is done by filtration. Preferably, the water content of the crystals obtained after the reduction of water is about 4% to about 6% by weight by KF. Preferably, the drying is conducted at a temperature of about 40° C. The drying step may be performed step-wise. Preferably, the drying step may be performed in two steps, wherein in the first step the crystals are dried under atmospheric pressure at a temperature of about 35° C. to about 45° C., preferably at a temperature of about 39° C. to about 41° C., more preferably at a temperature of about 40° C. for about 48 hours. In the second step, the crystals are dried under reduced pressure at a temperature of about 39° C. to about 41° C., preferably at a temperature of about 40° C., for about 72 hours. Preferably, the crystals are dried in a drying oven in an unsealed, closed glass vessel.

In one embodiment, the present invention provides a process for preparing pravastatin sodium Form B comprising suspending wet crystals of pravastatin sodium Form L in acetone, reducing the water to about 3% to about 7% by weight, and drying the crystals at a temperature of about 60° C. Preferably, the volume of acetone used is of about 15 times the mass of crystals to be suspended. Preferably, the reduction of the water content is done by filtration. Preferably, the crystals contain about 4% to about 6% of water by weight, prior to drying. The drying step may be done step-wise. Preferably, the drying step may be performed in two steps, wherein in the first step the crystals are dried under atmospheric pressure at a temperature of about 59° C. to about 61° C. for about 48 hours. In the second step of the two step drying process, the crystals are dried under reduced pressure at a temperature of about 59° C. to about 61° C. for about 72 hours. Preferably, the crystals are dried in an unsealed, closed glass vessel. Optionally, drying may be stopped after heating for 24 hours under atmospheric pressure at a temperature of about 60° C. Preferably, the crystals are dried at a temperature of about 60° C.

In another embodiment, the present invention provides a process for preparing pravastatin sodium Form B comprising providing dried crystals of pravastatin sodium Form D, suspending the dried crystals of Form D in a solvent mixture of water and acetone, reducing the water to about 3% to about 7% by weight, and drying the crystals. Preferably, the solvent mixture has a ratio of water to acetone of about 1 to 49 by volume, respectively. Form D is preferably suspended in the solvent mixture for about 20 hours. Preferably, the reduction of the water content is done by filtration. The crystals are preferably dried at a temperature of about 59° C. to about 61° C., more preferably at a temperature of about 60° C., under reduced pressure for about 24 hours. Preferably, the drying is performed on a glass plate placed in a drying oven.

In another embodiment, the present invention provides a process for preparing pravastatin sodium Form D comprising providing wet crystals of pravastatin sodium Form L, drying the wet crystals at a temperature of about 50° C. to about 70° C. under a pressure of between about reduced pressure to about atmospheric pressure.

Preferably, Form L is dried for about 24 hours at atmospheric pressure to form pravastatin sodium Form D.

Preferably, in an industrial scale, the drying process is done drop-wise. Preferably, the drying step may be performed in two steps, wherein the first step comprises drying the crystals until the water content of the crystals is about 3% to about 7% and heating the crystals for about 10 to about 12 hours, and thereafter drying the crystals in a second drying step. Preferably, the first drying step is performed at reduced pressure at a temperature of about 50° C. to about 63° C.; and thereafter, the crystals are heated at a temperature of about 65° C. to about 75° C. at atmospheric pressure. Form D may be present in about 20% by weight at the completion of the first drying step. Preferably, after the first drying step, a mixture of Form B and Form D is obtained. The second drying step is preferably performed under reduced pressure at a temperature of about 50° C. to about 70° C. Preferably, pravastatin sodium Form D formed by the production scale drying process of the invention contains less than about 2% water by weight.

One skilled in the art will recognize that the term “production scale drying process” conveys drying a large volume of material, such as 200 kg rather than 5 mg. One skilled in the art will also recognize that the scale of the reaction affects reaction parameters, such as heating and drying times.

In another embodiment, the present invention provides pharmaceutical compositions comprising the crystalline form of the present invention and at least one pharmaceutically acceptable excipient.

In another embodiment, the present invention provides pharmaceutical composition comprising pravastatin sodium of any of the forms: B, D or T made by the processes of the invention, and at least one pharmaceutically acceptable excipient.

The present invention further encompasses a process for preparing a pharmaceutical formulation comprising combining the pravastatin sodium of any of the forms: B, D or T made by the processes of the invention, with at least one pharmaceutically acceptable excipient.

The present invention further encompasses a process for preparing a pharmaceutical formulation comprising combining the crystalline form of the present invention with at least one pharmaceutically acceptable excipient.

The present invention further encompasses the use of the crystalline form of the present invention for the manufacture of a pharmaceutical composition.

The present invention further encompasses the use of pravastatin sodium of any of the forms: B, D or T made by the processes of the invention, for the manufacture of a pharmaceutical composition.

As used herein, the term “pharmaceutical compositions” or “pharmaceutical formulations” includes tablets, pills, powders, suspensions, emulsions, granules, capsules, suppositories, or injection preparations. Pharmaceutical compositions containing the crystalline form of the present invention, pravastatin sodium Form B or pravastatin sodium Form D may be prepared by using diluents or excipients such as fillers, bulking agents, binders, wetting agents, disintegrating agents, surface active agents, and lubricants. Various modes of administration of the pharmaceutical compositions of the invention can be selected depending on the therapeutic purpose, for example tablets, pills, powders, suspensions, emulsions, granules, capsules, suppositories, or injection preparations.

Any excipient commonly known and used widely in the art can be used in the pharmaceutical composition. Carriers used include, but are not limited to, lactose, white sugar, sodium chloride, glucose, urea, starch, calcium carbonate, kaolin, crystalline cellulose, silicic acid, and the like. Binders used include, but are not limited to, water, ethanol, propanol, simple syrup, glucose solutions, starch solutions, gelatin solutions, carboxymethyl cellulose, shelac, methyl cellulose, potassium phosphate, polyvinylpyrrolidone, and the like. Disintegrating agents used include, but are not limited to, dried starch, sodium alginate, agar powder, laminalia powder, sodium hydrogen carbonate, calcium carbonate, fatty acid esters of polyoxyethylene sorbitan, sodium laurylsulfate, monoglyceride of stearic acid, starch, lactose, and the like. Disintegration inhibitors used include, but are not limited to, white sugar, stearin, coconut butter, hydrogenated oils, and the like. Absorption accelerators used include, but are not limited to, quaternary ammonium base, sodium laurylsulfate, and the like. Wetting agents used include, but are not limited to, glycerin, starch, and the like. Adsorbing agents used include, but are not limited to, starch, lactose, kaolin, bentonite, colloidal silicic acid, and the like. Lubricants used include, but are not limited to, purified talc, stearates, boric acid powder, polyethylene glycol, and the like. Tablets can be further coated with commonly known coating materials such as sugar coated tablets, gelatin film coated tablets, tablets coated with enteric coatings, tablets coated with films, double layered tablets, and multi-layered tablets.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, and semisynthesized glycerides.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic.

Additional ingredients, such as dissolving agents, buffer agents, and analgesic agents may be added. If necessary, coloring agents, preservatives, perfumes, seasoning agents, sweetening agents, and other medicines may also be added to the desired preparations.

The amount of the crystalline form of the present invention, pravastatin sodium Form B or pravastatin sodium Form D contained in a pharmaceutical composition for treating atherosclerosis or hypercholesterolemia should be sufficient to treat or ameliorate atherosclerosis or hypercholesterolemia.

The pharmaceutical compositions of the invention may be administered in a variety of methods depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations may be administered intramuscularly, intracutaneously, subcutaneously or intraperitoneally. Suppositories may be administered into the rectum.

The dosage of a pharmaceutical composition containing the crystalline form of the present invention, pravastatin sodium Form B or pravastatin sodium Form D for treating atherosclerosis or hypercholesterolemia according to the invention will depend on the method of use, the age, sex, and condition of the patient.

Having described the invention with reference to certain preferred embodiments, other embodiments will become apparent to one skilled in the art from consideration of the specification. The invention is further defined by reference to the following examples describing in detail the synthesis of pravastatin sodium polymorphs by production scale drying methods and methods for preparing the crystalline form of the present invention. It will be apparent to those skilled in the art that many modifications, both to materials and methods, may be practiced without departing from the scope of the invention.

### EXAMPLES

X-ray powder diffraction data were obtained using an ARL powder X-ray diffractometer model X′TRA-030 equipped with a Peltier detector and copper radiation of 1.5418 Å. Samples were scanned at a scanning speed of 3°/min. A round aluminum sample holder with zero background quartz plate was used. All peak positions are within ±0.1 degrees two theta.

Differential scan calorimetry (DSC) analysis was performed using a Mettler Toledo 822^{e}/700 differential scanning calorimeter. The weight of the samples was about 3 mg to about 5 mg. The samples were scanned at a rate of 10° C./min from 30° C. to at least 250° C. The oven was constantly purged with nitrogen gas at a flow rate of 40 ml/min. Standard 40 μl aluminum crucibles covered by lids with 3 holes were used.

Thermogravimetric analysis (TGA) was performed using a Mettler Toledo 851^{e} thermogravimeter. The samples weighed about 7 mg to about 15 mg and were scanned at a rate of 10° C./min from 25° C. to 250° C. The oven was constantly purged with nitrogen gas at a flow rate of 50 ml/min. Standard 150 μl alumina crucibles covered by lids with 1 hole were used.

Karl Fisher analysis was performed according to methods well known in the art.

FT-IR was obtained in KBr pellet and a Nujol mull using a Perkin Elmer Spectrum 1000 spectrometer at 4 cm⁻¹ resolution with 16 scans, in the range of 4000-400 cm⁻¹ or 4000-600 cm⁻¹.

### Example 1

### Preparation of Wet Pravastatin Sodium Form L

Pravastatin sodium was crystallized from a solvent mixture of water and acetone where the water to acetone ratio was about 1:16 by volume. Thereafter, the pravastatin sodium crystals were filtered and washed with a solvent mixture of water and acetone in a ratio of 1:49 by volume, and then washed with pure acetone. The crystals contained from about 11% to about 15% water by weight as determined by Karl Fischer analysis. The crystals contained about 40% to about 50% of pravastatin sodium by weight as determined by loss on dry, and acetone. The resulting crystals were determined to be pravastatin sodium Form L by XRD.

### Example 2

### Making Pravastatin Sodium Form D from Form L

Wet crystals of pravastatin sodium Form L were dried on a glass plate in a laboratory drying oven at atmospheric pressure at about 50° C. to about 70° C. for about 24 hours. The resulting crystals were determined to be pravastatin sodium Form D by XRD.

### Example 3

### Production Scale Drying Synthesis of Pravastatin Sodium Form D and Form B From Form L

The process was run on a scale to obtain about 200 kg of dried pravastatin sodium. Wet crystals of pravastatin sodium Form L were dried under reduced pressure of about 76.0 mm Hg until the water content of the crystals was determined by Karl Fischer analysis to be about 3% to about 7% by weight. The resulting crystals were determined to be a mixture of pravastatin sodium Form D and Form B by XRD.

### Example 4

### Production Scale Drying of Pravastatin Sodium to Obtain Pravastatin Sodium Form D

The process was run on a scale to obtain about 200 kg of dried pravastatin sodium. Wet crystals of pravastatin sodium Form L were dried under pressure of about 76.0 mm Hg at about 50° C. to about 63° C. until the water content of the crystals was determined by Karl Fischer analysis to be about 3% to about 7% by weight. The crystals were then heated at 70° C. for 10 to 12 hours under atmospheric pressure. The crystals were thereafter dried under reduced pressure of about 76.0 mm Hg at a temperature of about 50° C. to about 70° C. until the water content of the crystals was less than about 2% as measured by Karl Fischer analysis. The resulting crystals were determined to be pravastatin sodium Form D by XRD.

### Example 5

### Synthesizing Pravastatin Sodium B from Form L

Wet crystals of pravastatin sodium Form L were suspended for about ten hours at room temperature in a volume of acetone measuring about 15 times the amount of crystals used. The suspended crystals were then filtered until the water content of the crystals measured about 5.6% by weight by Karl Fischer analysis. The crystals were placed into an unsealed, closed glass vessel and dried in a laboratory drying oven under atmospheric pressure at about 60° C. for about 48 hours. Thereafter, drying continued under reduced pressure at about 60° C. for about 72 hours.

XRD performed after drying for twenty-four hours at atmospheric pressure revealed that pravastatin sodium Form B was present. The resulting crystals were determined to be pravastatin sodium Form B by XRD.

### Example 6

### Synthesizing Pravastatin Sodium B from Form L

Wet crystals of pravastatin sodium Form L were suspended for about ten hours at room temperature in a volume of acetone measuring about 15 times the amount of crystals used. The suspended crystals were then filtered until the water content of the crystals measured about 4.6% by weight by Karl Fischer analysis. The crystals were placed into an unsealed, closed glass vessel and dried in a laboratory drying oven under atmospheric pressure at about 60° C. for about 48 hours. Thereafter, drying continued under reduced pressure at about 60° C. for about 72 hours.

An XRD performed after drying at 60° C. at atmospheric pressure for 24 hours revealed that pravastatin sodium Form B was present. The resulting crystals were determined to be pravastatin sodium Form B by XRD.

### Example 7

### Method of Making the Crystalline Form of the Present Invention

Wet crystals of pravastatin sodium Form L were suspended for about ten hours at room temperature in a volume of acetone measuring about 15 times the amount of crystals used. The suspended crystals were then filtered until the water content of the crystals measured about 3% to about 7% by weight as determined by Karl Fischer analysis. The crystals were placed into an unsealed, closed glass vessel and dried in a laboratory drying oven at about 40° C. for about 48 hours under atmospheric pressure. Thereafter, drying continued at about 40° C. for about 72 hours under reduced pressure.

An XRD performed after drying at 40° C. under atmospheric pressure for 24 hours revealed that pravastatin sodium Form G was present. The resulting crystals were determined to be the crystalline form of the present invention by XRD.

### Example 8

### Synthesizing Pravastatin Sodium Form B from Form D

Dried crystals of pravastatin sodium Form D were suspended in a solvent mixture of water and acetone wherein the water to acetone ratio was about 1:49 by volume for about 20 hours. The suspended crystals were then filtered until the crystals contained about 3.1% water by weight as determined by Karl Fischer analysis. The crystals were thereafter dried on a glass plate in a laboratory drying oven at 60° C. under reduced pressure for about 24 hours. The resulting crystals were determined to be pravastatin sodium Form B by XRD.

1. A crystalline form of pravastatin sodium characterized by data selected from the group consisting of: an X-ray powder diffraction with peaks at 3.3, 3.9, 5.4, 6.4, 16.8, and 17.5±0.1 degrees two-theta, an FT-IR spectrum with peaks at 1157, 1181, 1570, and 1731±2 cm⁻¹ and DSC thermogram having a broad endotherm at about 108° C., and an endotherm at about 176° C.

2. The crystalline form of claim 1, wherein the crystalline form is a hydrate.

3. The crystalline form of claim 2, wherein the crystalline form is a monohydrate.

4. The crystalline form of claim 1, having a water content of about 2.8% to about 4.2% by weight, as measured by Karl Fisher or TGA.

5. The crystalline form of claim 2, having a water content of about 3.8%.

6. The crystalline form of claim 1, characterized by an X-ray powder diffraction peaks at 3.3, 3.9, 5.4, 6.4, 16.8, and 17.5 degrees two-theta, ±0.1 degrees two-theta.

7. The crystalline form of claim 6, wherein the crystalline form is further characterized by X-ray powder diffraction peaks at 10.2, 13.8, 18.0, 19.3, 19.5, and 21.8 degrees two-theta, ±0.1 degrees two-theta.

8. The crystalline form of claim 7, having an XRD pattern substantially as depicted in FIG. 1.

9. The crystalline form of claim 1, characterized by an FT-IR spectrum with peaks at 1157, 1181, 1570, and 1731±2 cm⁻¹.

10. The crystalline form of claim 9, wherein the crystalline form is further characterized by an FT-IR spectrum with peaks at 722, 823, 843, 964, 1014, 1037, 1080, 1109, and 1263±2 cm⁻¹.

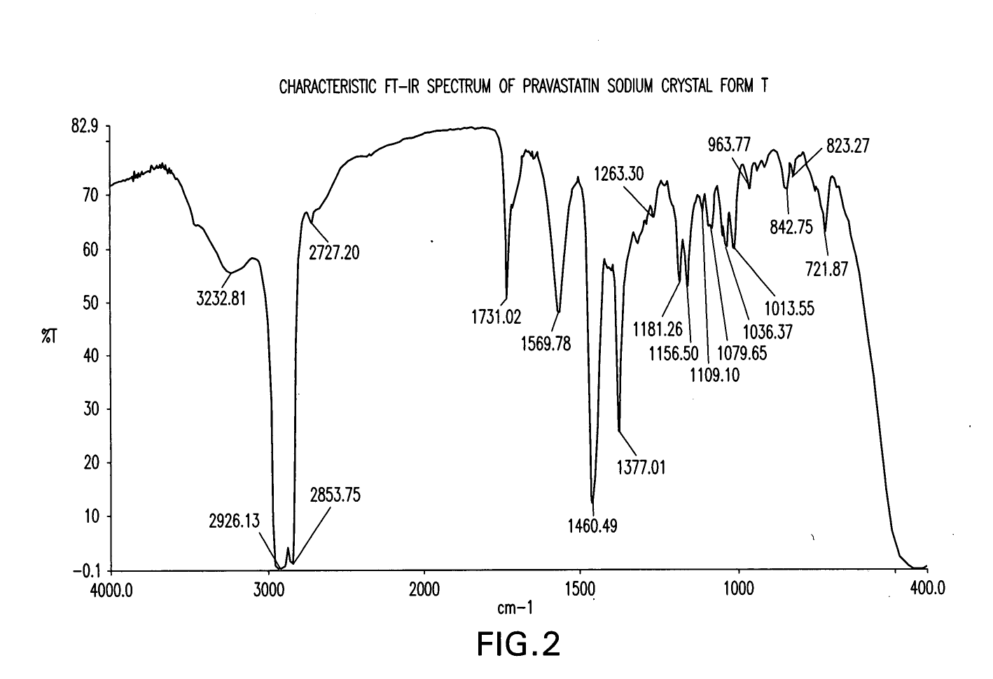

11. The crystalline form of claim 10, having an FTIR spectrum substantially as depicted in FIG. 2.

12. The crystalline form of claim 1, characterized by DSC thermogram having a broad endotherm at about 108° C., and an endotherm at about 176° C.

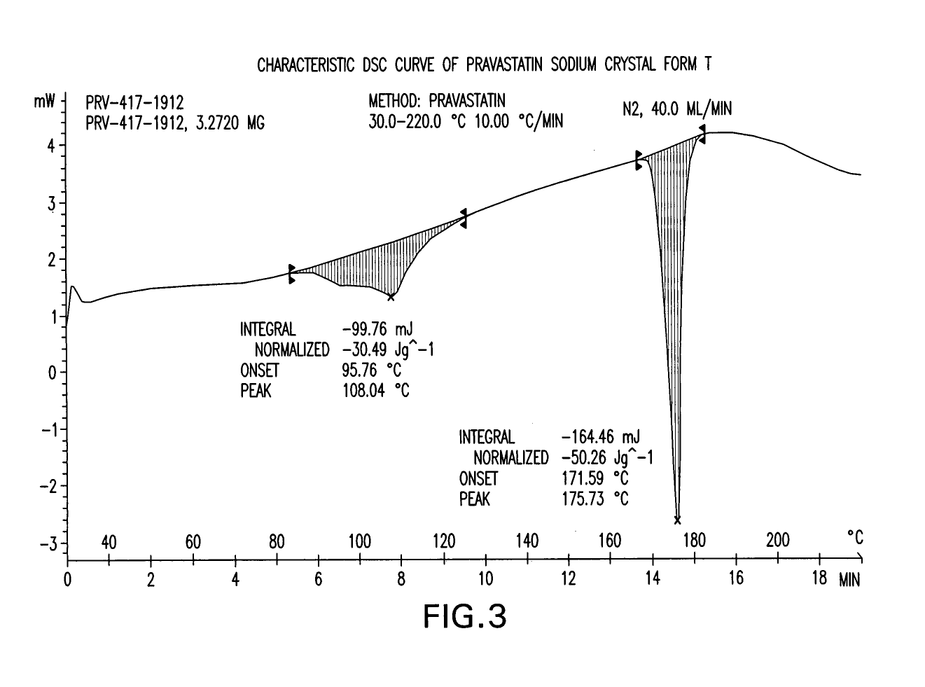

13. The crystalline form of claim 12, having a DSC thermogram substantially as depicted in FIG. 3.

14. The crystalline form of claim 1, having less than about 10% by weight of other crystal forms.

15. The crystalline form of claim 1, having particle size of less than about 250 μm.

16. The crystalline form of claim 1, wherein the crystals have an irregular shaped stacked plates and fractured edges and a rough surface.

17. A process for preparing pravastatin sodium of claim 1, comprising suspending wet crystals of pravastatin sodium Form L in acetone, reducing the water content to about 3% to about 7% by weight, and drying the crystals at about 35° C. to about 45° C. to obtain pravastatin sodium of claim 1.

18. The process of claim 17, wherein the volume of acetone used is of about 15 times the mass of crystals to be suspended.

19. The process of claim 17, wherein the reduction of the water content is done by filtration.

20. The process of claim 17, wherein the water content of the crystals obtained after the reduction of water is of about 4% to about 6% by weight.

21. The process of claim 17, wherein the drying is conducted at a temperature of about 40° C.

22. The process of claim 17, wherein the drying is done step-wise.

23. The process of claim 22, wherein the drying is done in two steps wherein in the first step the crystals are dried under atmospheric pressure at a temperature of about 35° C. to about 45° C. for about 48 hours, and in the second step, the crystals are dried under reduced pressure at about 39° C. to about 41° C. for about 72 hours.

24. The process of claim 22, wherein the first drying step is at a temperature of about 39° C. to about 41° C.

25. The process of claim 24, wherein the first drying step is at a temperature of about 40° C.

26. The process of claim 22, wherein the second drying step is at a temperature of about 40° C.

27. A process for preparing pravastatin sodium Form B comprising suspending wet crystals of pravastatin sodium Form L in acetone, reducing the water to about 3% to about 7% by weight, and drying the crystals at about 60° C.

28. The process of claim 27, wherein the volume of acetone used is of about 15 times the mass of crystals to be suspended.

29. The process of claim 27, wherein the reduction of the water content is done by filtration.

30. The process of claim 27, wherein the crystals contain about 4% to about 6% of water by weight, prior to drying them.

31. The process of claim 27, wherein the drying is done step-wise.

32. The process of claim 31, wherein the drying is in two steps, wherein in the first step the crystals are dried under atmospheric pressure at a temperature of about 59° C. to about 61° C. for about 48 hours, and in the second step the crystals are dried under reduced pressure at about 59° C. to about 61° C. for about 72 hours.

33. The process of claim 31, wherein the crystals are dried at a temperature of about 60° C.

34. A process for preparing pravastatin sodium Form B, comprising providing dried crystals of pravastatin sodium Form D, suspending the dried crystals of Form D in a solvent mixture of water and acetone, reducing the water to about 3% to about 7% by weight, and drying the crystals.

35. The process of claim 34, wherein the ratio of water to acetone is of about 1 to 49 by volume, respectively.

36. The process of claim 34, wherein Form D is suspended in the solvent mixture for about 20 hours.

37. The process of claim 34, wherein the reduction of the water content is done by filtration.

38. The process of claim 34, wherein the crystals are dried at a temperature of about 59° C. to about 61° C. under reduced pressure for about 24 hours.

39. The process of claim 38, wherein the crystals are dried at a temperature of about 60° C.

40. The process of claim 34, wherein the drying is performed on a glass plate placed in a drying oven.

41. A process for preparing pravastatin sodium Form D comprising providing wet crystals of pravastatin sodium Form L, drying the wet crystals at a temperature of about 50° C. to about 70° C. under a pressure of between about reduced pressure to about atmospheric pressure.

42. The process of claim 41, wherein Form L is dried for about 24 hours at atmospheric pressure to form pravastatin sodium Form D.

43. The process of claim 41, wherein the process is done in an industrial scale.

44. The process of claim 43, wherein the drying process is done step-wise.

45. The process of claim 44, wherein the drying process is done in two steps wherein the first step comprises drying the crystals until the water content of the crystals is about 3% to about 7% and heating the crystals for about 10 to about 12 hours, and thereafter drying the crystals in a second drying step.

46. The process of claim 45, wherein the first drying step is performed at reduced pressure at a temperature of about 58° C. to about 63° C.; and thereafter the crystals are heated at a temperature of about 50° C. to about 70° C. at atmospheric pressure.

47. The process of claim 45, wherein after the first drying step, a mixture of Form B and Form D is obtained.

48. The process of claim 44, wherein the second drying step is performed under reduced pressure at a temperature of about 50° C. to about 70° C.

49. The process of claim 44, wherein the obtained pravastatin sodium Form D contains less than about 2% water by weight.

50. Pharmaceutical compositions comprising pravastatin sodium of claim 1 and at least one pharmaceutically acceptable excipient.

51. Pharmaceutical composition comprising pravastatin sodium of any of the forms: B, D or pravastatin sodium of claim 1 made by the processes of the invention, and at least one pharmaceutically acceptable excipient.

52. A process for preparing a pharmaceutical formulation comprising combining the pravastatin sodium of claim 1 with at least one pharmaceutically acceptable excipient.

53. A process for preparing a pharmaceutical formulation comprising combining the pravastatin sodium of any of the forms: B, D or pravastatin sodium of claim 1 made by the processes of the invention, with at least one pharmaceutically acceptable excipient.

54. Use of pravastatin sodium of claim 1 for the manufacture of a pharmaceutical composition.

55. Use of pravastatin sodium of any of the forms: B, D or pravastatin sodium of claim 1 made by the processes of the invention, for the manufacture of a pharmaceutical composition.
